(19) United States
(12) Patent Application Publication
(10) Pub. No.: US 2006/0009726 A1
(43) Pub. Date: Jan. 12, 2006

(54) Protective enclosure for body support

(76) Inventor: Margarita Clement, Northridge, CA (US)

Correspondence Address: MARGARITA CLEMENT, 17537 BLYTHE STREET, NORTHRIDGE, CA, 91325, US

(21) Appl. No.: 11/227,792

(22) Filed: Sep. 15, 2005

Related U.S. Application Data
(63) Continuation-in-part of application No. 09/063,985, filed on Apr. 21, 1998, now Pat. No. 6,443,920.

(51) Int. Cl. A61F 13/00; A61F 13/06

(52) U.S. Cl. 602/61; 602/60; 602/62

(57) ABSTRACT

A soft, flexible, low friction human protective enclosure designed to prevent occurrences of skin deterioration by cushioning body prominences for bedridden or wheelchair bound patients and to promote ease of ambulation. A support wrap (10) is made of sturdy, flame-retardant durable low friction fabric material for ease of moving enclosed patient along the surface of bed or chair. An inner filler made of soft and pliable fibrous or rubber-like composite material is enveloped by the support wrap (10). Velcro® latches (30) and (40) and transfer belts (55) at predetermined locations provide means for attachment and adjustment when the support is closed around the body.

This application is a Continuation In-Part Application (CIP) of application Ser. No. 09/063,985 filed Apr. 21, 1998, now U.S. Pat. No. 6,443,920 B1 Granted on Sep. 3, 2002.

References Cited

U.S. Patent Documents:

| | | | | |
|---|---|---|---|---|
| 2,531,757 A | * | 11/1950 | Whinery | 602/79 |
| 4,084,586 A | * | 4/1978 | Hettick | 602/60 |
| 4,474,573 A | * | 10/1984 | Kinchenberg et al. | 602/19 |
| 4,832,010 A | * | 5/1989 | Lerman | 602/63 |
| 4,926,845 A | * | 5/1990 | Harris | 602/19 |
| 4,977,898 A | * | 12/1990 | Hunt | 602/61 |
| 5,423,852 A | * | 6/1995 | Daneshvar | 606/201 |
| 5,425,702 A | * | 6/1995 | Carn et al. | 602/62 |
| 5,595,192 A | * | 1/1997 | Tatum | 128/869 |
| 5,706,523 A | * | 1/1998 | Wetzel | 2/238 |
| 5,779,658 A | * | 7/1998 | Saca | 602/61 |
| 5,484,395 | * | 1/1996 | DeRoche | 602/19 |
| 5,647,378 | * | 7/1997 | Farnum | 128/876 |
| 5,814,001 | * | 9/1998 | Schwenn et al. | 602/24 |
| 5,928,175 | * | 7/1999 | Tanaka | 602/75 |

* Cited by examiner

Primary Examiner—Kim M. Lewis

### CROSS-REFERENCE TO RELATED APPLICATION

Refer to Page No.: 1

### FEDERALLY SPONSORED RESEARCH

Not Applicable

### SEQUENCE LISTINGS OR PROGRAMS

Not Applicable

### BACKGROUND OF THE INVENTION

1. Field of Invention

This invention relates to a protective device of soft, pliable material designed to provide cushioning of bony prominences of bedridden or wheelchair bound patients and to promote safer ambulation.

2. Background of Invention

For cushioning and protective purposes, it is already well known to utilize pull-on and soft-wrap support devices to protect elbows and knees from abrasion, and to restrict movements of inflamed joints.

As intended, these devices prevent bruising from falls, and ease pain from swollen joints. However, patients have been unable to find a protective support device to alleviate occurrences of bedsores, or Decubitis (localized area of inflammation or deterioration of skin and subcutaneous tissue produced by pressure points).

Although knee and elbow supports provide protection to those specific areas, they are relatively useless in cases involving bedridden, wheelchair bound or immobile patients suffering from Decubitis. They do not provide therapeutic relief to pressure exerted on the skin between bony prominences, e.g. hips, lower back, buttocks, coccyx, knees and the surface on which the body rests (mattress, examination table, wheelchair, etc.).

Currently, physicians order a topical medication and pads for the affected areas. However, these dressings cover only the inflamed areas while failing to prevent similar skin deterioration elsewhere on the body. This present invention provides protection of the hips, buttocks, coccyx, lower back, and other pressure points from the abdominal region down to knees in the form of a formable, soft, pliable cushion of predetermined configuration and prevents irritation such as bedsores caused by movement of the patient for purposes of comfort or administering personal hygiene.

A single transfer belt is currently being used for ambulating patients. This method is not safe because the assistant/caregiver does not have total control over the patient's balance while walking. The patient's upper torso can easily tilt forward over the belt. The single belt is also constricting the abdominal region, making the ambulating uncomfortable to the patient.

The design of my invention helps to solve this problem The patient is enclosed in the protective device while being ambulated, whereas the plurality of transfer belts provide for better control of balance preventing the patient's upper torso filling forward.

### SUMMARY OF THE INVENTION

Empirical studies and prior literature provide sufficient evidence to conclude that Decubitis, bedsores, and inflamed tissue pose serious problems to persons confined to bed for prolonged periods or whose incapacitation prevent voluntary movements.

The problem arises from (a) contact with firm bed mattresses or wheelchair seats and (b) by infrequent rotation of the patient for long periods. In most cases, the deterioration of skin occurs in the area of bony prominences at the hips, buttocks and knees where there is insufficient subcutaneous tissue, and, to a lesser extent, the upper back or shoulders where the body makes contact with the bed or other devices to rest upon.

While physicians order topical medication and pads for the affected areas, these dressings cover only those inflamed areas while failing to prevent similar ulcerated areas elsewhere. The enclosing body support will protect bony prominences from the lower torso to knees vulnerable to Decubitis.

The design of my invention allows for the placement of the individual on the device while it is open and in flat configuration. The support is then simply closed with adjustable Velcro® tape latches, which facilitate proper fit and comfort.

The device is also designed with an opening in the perineal area to provide for urination or secretion without having to remove the device from the patient. For purpose of bathing or personal hygiene, the Velcro® tape latches are released and the enclosure is opened to allow free access to the patient without discomfort.

The objective and advantages of the body support wrap of the present invention are:
(a) to relieve pain and discomfort from inflammation and remove pressure to the skin from bony prominences, with particular attention to hips, buttocks, coccyx, lower extremity pressure points and knees;
(b) to keep the affected areas warm, thus stimulating blood circulation for bedridden or wheelchair bound patients unable to move frequently;
(c) to keep the skin protected from abrasive material such as starched sheets, mattress covers, and the residual laundry chemicals left on the sheets or garment;
(d) that the patient, when able, can attach or remove the enclosure with ease by himself/herself without assistance;
(e) that the device allows for movement of patient (rotation in bed) using the device transfer belts instead of directly grasping the patient's extremities or clothing;
(f) that the patient may also move to and from bed or chair with minimal discomfort without having to reposition or re-fit the device;
(g) that the device permits ease of access through opening of areas requiring attention to personal hygiene, urination and secretion, without removing the device;
(h) that the device is provided with detachable 24 hours monitoring labels to allow for permanent records of recommended patient's turning required by law.

Additional objectives and advantages are to provide a protective enclosure which can be easily and conveniently opened and closed with minimal discomfort to the patient, thereby increasing muscular, skin and vascular tone through unrestricted blood circulation. Among other advantages, the present invention eliminates the need for rubber rings or doughnuts that merely increase the pressure around bony prominences. The present device protects major bony prominences from abdominal region to knees in contact with mattress or chair simultaneously, yet allows the patient to freely move (rotate or sit) with the device attached to the body. This device does not discriminate based on a patient's sex or age, and any patient with potential susceptibility to skin deterioration can use it.

The protective enclosure with transfer belts provides safety in assisting, turning and repositioning the patient while in bed, chair or wheelchair as well as promotes safer ambulation. The flexibility of the device allows for changes in body posture when such change is desired for increased comfort. The transfer belts provide added safety for patient with restricted mobility who may be at risk of falling and promote added safety in body mechanics for nurses, caregivers or family members by assisting them in transferring the patient without having to grasp the patient's extremities. The transfer belts double loop ensures security against inadvertent opening or tampering of the device.

In summary, the present invention provides an enclosure for added comfort to bedridden and wheelchair bound patients which also serves to promote ease of ambulation.

### BRIEF DESCRIPTION OF THE DRAWINGS

The present invention will be better understood by reference to the drawings wherein:

FIG. 1 is a plan view of the protective enclosure for body support 10 according to the present invention while open, showing support wraps for the abdominal region 15 and each leg 25 and 27 sewn together at two specific places 45. It also shows the extra detachable pads 20 for additional comfort, placed as needed at affected areas.

FIG. 2 is an outside plan view showing the protective enclosure for body support 10 with transfer belts 55, fastening loops 57 and belt loops 60.

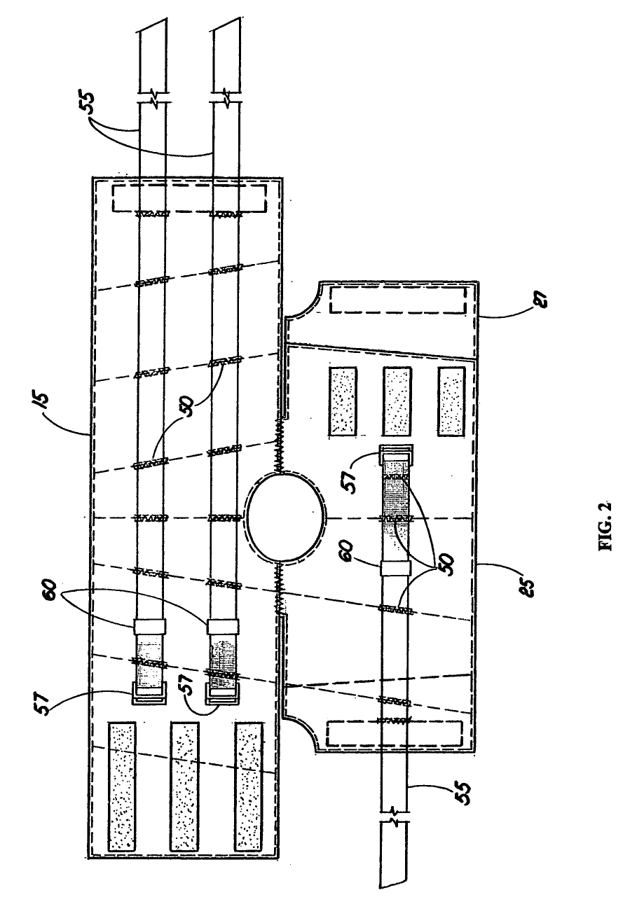

FIG. 3 is a perspective view showing the protective enclosure for body support 10 as it would appear when wrapped around a patient's body, cushioning the bony prominences of the abdominal region, hips, thighs and knees.

### DETAILED DESCRIPTION

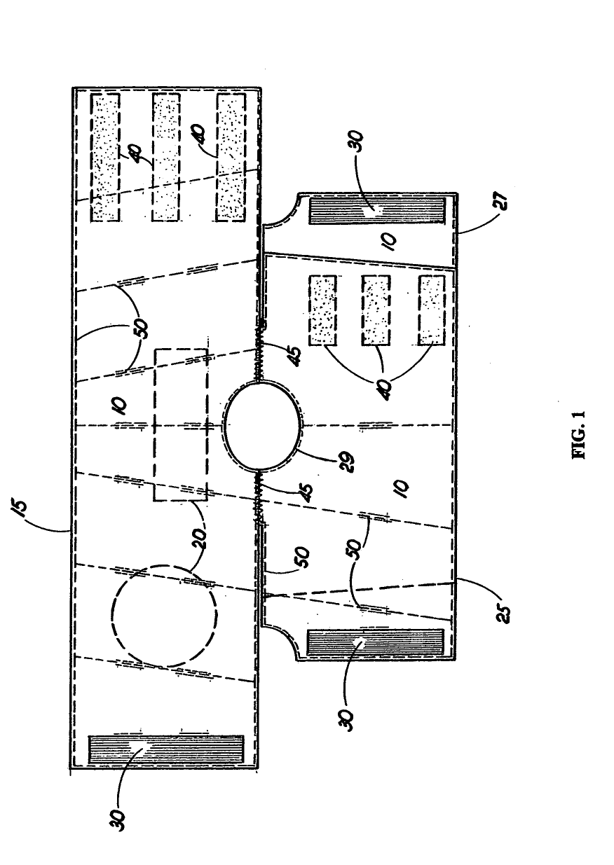

A preferred embodiment of the present invention is illustrated in FIG. 1. The protective enclosure for body support device 10 comprises three parts sewn together at two specific places 45. An abdominal region support wrap of rectangular shape 15 and a support wrap of slightly irregular rectangular shape for each leg 25 and 27 overlapping each other as shown in the FIG. 1. The outer lining is made of sturdy fabric material as nylon, rayon, acrylic, cotton, Dacron®, polyester, or various fabric blends.

The filler is made of soft and pliable composite material such as polyester fiber, acrylic fiber, foam rubber (flat sheet, egg-shaped, aerated, etc.) or any other soft and pliable material, providing a cushioning effect of preventing contact between the patient's bony prominences and the bed or chair, and to allow inflamed areas of the patient's body to breathe and benefit from increased blood circulation to existing skin lesions or bedsores.

The wraps are sewn around the perimeter and several other places with thread 50 of suitable material compatible to the material of the outer lining.

For additional comfort one or more detachable pads 20 of various sizes and shapes (round, rectangular, oblong, etc.) could be added to the device at locations of the body where extra padding is needed to further cushion body prominences of the patient. The pads will be manufactured from the same specified material as for body wraps outer lining and filler. The pads shall be attached to support wraps on the inside by means of peel-off double sided adhesive tape or by any other suitable means.

For sanitary reasons, the external fabric of support wraps 15, 25 and 27, and detachable pads 20 can be made also from disposable suitable material in order to make the detachable pads and the entire body support disposable.

The rectangular abdominal region support wrap 15 and the legs supporting wrap 25 and 27 can be made also with rounded or beveled corners to facilitate ease of manufacturing.

For the purpose of administering personal hygiene or urination and secretion, an opening 29, as cutout in the wraps is provided.

Velcro® latches or fasteners 30 and 40 are sewn to the support wraps to facilitate opening and closing of the device as well as to allow for adjustment to body size and patient's comfort.

The Velcro® latch or fastener comprises a hook tape 30 and loop tape 40. The loop tapes 40 are made longer to permit adjustability of support wrap for comfortable proper fit to the patient's body. A plurality of Velcro® loop tapes 30 will vary according to the size of the enclosure device.

For an added safety in ambulating a transfer belts 55 made of seat belt material are provided. Two belts on the abdominal wrap and one on each leg wrap. The belts are secured by a double loops 57 to ensure against opening and tampering by the patient. The ends of belts are passed through belt loops 60.

A size of present invention will vary with the size of the patient: small, medium and large. The sizes also accommodate variations in accordance with sex, age, and body measurements.

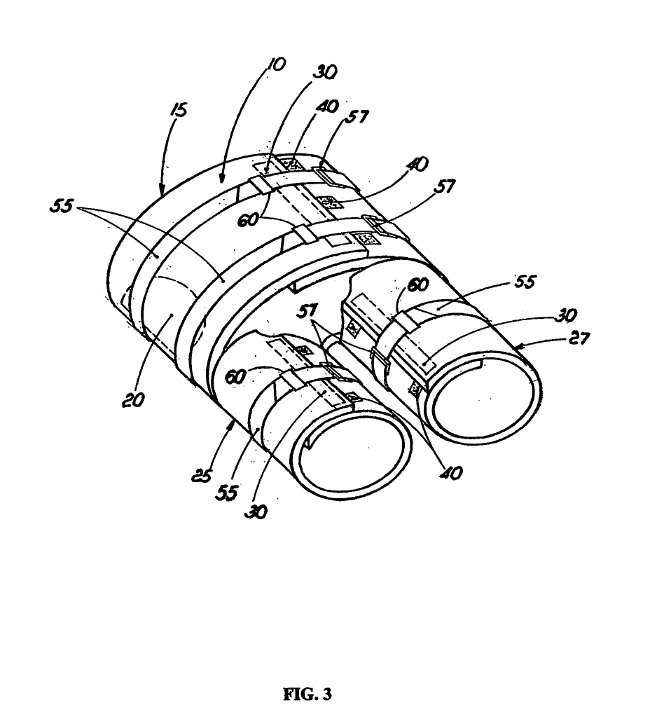

A perspective view of the present invention is shown in FIG. 3, illustrating the enclosure body support as it would appear when closed around a patient's body by Velcro® fasteners and transfer belts.

Additional advantages of the protective enclosure for body support wrap 10 include:
(a) a bedridden or wheelchair bound patient will be relieved of pain caused by hard contact with mattresses and seats;
(b) the cushioning effect will prevent development of inflamed tissue around bony prominences and allows the curative effect of increased blood circulation to existing lesions or bedsores;
(c) the Velcro® fasteners will permit quick and easy opening and closing of the device for purpose of bathing, changing undergarments, personal hygiene, and application of topical medication or new dressings.

The manner of using the protective enclosure for body support is simple and convenient, either for the attending health care provider, family member or for the patient himself/herself. The patient is placed with the back on the open device so that the lower body portion of the patient's torso rests on the abdominal wrap 15. The patient is then centered on the abdominal wrap 15 with the buttocks and coccyx slightly above the opening 29 and with his legs overlying the legs wraps 25 and 27. The protective enclosure for body support 10 is then closed thereby covering the body from the abdominal region to the knees with the inner lining protecting and cushioning the bony prominences and at the same time a cutout 29 permits catheterization, urination and secretion.

When the wrap is closed, the device is held in place on the patient's body by the Velcro® latches 30 and 40, adjusted for body size and patient's comfort. Transfer belts 55 shall be used for ambulating, turning and repositioning only. Opening of the device is equally simple and easy by pulling the Velcro® hook tape 30 and loop tape 40 apart to free the overlapping edges of the support.

The present invention thus protects and supports the body easily and conveniently, can be closed and reopened as often as needed as treatment or hygiene requires, and can be removed without discomfort to the individual. The invention thus relieves pressure on skin and subcutaneous tissue; prevents sensory loss, or absence of the patient's awareness of pain and pressure; permit's the supply of nutrients to tissue cells, thus avoiding edema, by improving blood circulation which, in turn, aids the healing of existing ulcers or bedsores; and facilitates increased activity by the patient because the device reduces or eliminates the discomfort of motion, allowing activity to thereby increase muscular, skin, and vascular tone.

What is claimed is:

1. A protective enclosure for body support to prevent skin deterioration by cushioning patient's body from waist to knees, comprising:
(a) an enclosing rectangular abdominal support wrap and a slightly irregular rectangular support wrap for each leg of predetermined configuration, sewn together at two specific places;
(b) an inner filer of soft pliable material enveloped by outer lining to cushion critical area of the said body region;
(c) one or more detachable pads of various shapes for additional cushioning to affected areas;
(d) a plurality of loop and hook tape fasteners of predetermined size placed at specific locations on the outer surface of support wraps to permit the closure of the body support;
(e) a plurality of transfer belts attached to the abdominal and leg wraps, to assist in ambulating, turning, transferring and repositioning of patient;
(f) an opening in the support wraps for the purpose of administering personal hygiene.

2. A protective enclosure for body support according to claim 1(b) wherein the said inner filler material is made of soft and pliable composite material, such as polyester fiber, acrylic fiber, various foam rubber flat sheets or other fibrous materials of predetermined specific density to provide for effective cushioning.

3. A protective enclosure for body support according to claim 1(b) wherein the said outer lining is made of sturdy low friction material such as nylon, rayon, acrylic, cotton, polyester, or other suitable fabric blends.

4. A protective enclosure for body support according to claim 1(a) wherein the corners of said rectangular shape support wraps are rounded or beveled to facilitate manufacturing.

5. A protective enclosure for body support according to claim 1(d) wherein the said loop fastening tape is longer than the hook tape to permit adjustment of the size of the support, when it is wrapped around the patient's body.

6. A protective enclosure for body support according to claim 3 whereas the said outer lining material of support wraps and of the said detachable pads of claim 1(c) are, for sanitary reasons, made of suitable disposable material.

7. A protective enclosure for body support provided with left and right 24 hour monitoring peel-off labels to allow for permanent records.
